(19) United States
(12) Patent Application Publication
(10) Pub. No.: US 2019/0112332 A1
(43) Pub. Date: Apr. 18, 2019

(54) Protein Electrodialiysis (PED) Device for Enrichment of the Extracellular Proteins Deposited by Microorganisms in Liquid Culture

(71) Applicant: Abolfazl Lotfi, Tehran (IR)

(72) Inventor: Abolfazl Lotfi, Tehran (IR)

(21) Appl. No.: 15/784,140

(22) Filed: Oct. 15, 2017

(51) Int. Cl. C07K 1/36; C12M 1/00; C07K 1/24; C07K 1/34; B01D 61/42

(52) U.S. Cl. CPC C07K 1/36; C12M 47/10; B01D 2313/06; C07K 1/34; B01D 61/422; C07K 1/24

(57) ABSTRACT

An efficient tool with low cost to rapidly isolate and concentrate the secreted proteins of microorganisms in high quality. By this device, the secreted proteins are concentrated by passing through a dialysis membrane at electrophoretic conditions.

### BACKGROUND OF INVENTION

Microorganisms such as filamentous fungi and bacteria produce secreted proteins and enzymes through dispensable metabolic pathways. So far, several methods have been described for isolation and enrichment of the secreted proteins from cultures of microorganisms. These methods can be readily applied to study differential protein expression and metabolic pathways in microorganisms by proteomic approaches. The purpose of Protein Enrichment Methods is to obtain distinguishable protein fractions and increase the concentration of the proteins of interest. Several methods are commonly used for protein enrichment that the main ones have been listed as follows:

### A: Centrifugation

Centrifugation is considered as the simplest method for protein enrichment and fractionation. It can be used to separate cell substructures in cases where the protein of interest is locally concentrated (e.g. in the membrane, mitochondria or nucleus). By using this method, you can effectively separate the cellular homogenate into its individual components based on molecular weight, size and shape.

Centrifugation can also be used to fractionate protein mixtures based on their coefficient of sedimentation. Basically, the smaller the coefficient value (expressed in Svedberg units), the slower the molecule moves in a centrifugal field. Upon centrifugation, the protein particles will separate based on protein density, mass and shape. It is possible to further enhance the efficiency of this step with gradient centrifugation (Sharma et al. 2010; Jiang et al. 2011).

### B: Precipitation

Proteins can also be purified and enriched using precipitation technique. In this method, high amount of a neutral salt (e.g. ammonium sulphate, sodium chloride) is added to the solution to enhance the interactions which consequently leads to the aggregation and precipitation of proteins (Bodzon-Kulakowska et al. 2007).

Generally, ammonium Sulphate can precipitate most proteins at saturation (3.9 M at 0° C. or 4.04 M at 20° C.) while preventing bacterial growth and denaturation. The salting-out process can also be manipulated to facilitate selective protein separation by simply varying the salt concentration.

A similar approach is to use a dialysis technique. The dilute protein solution is placed in a dialysis bag or alternative dialysis device, such as Tube-O-DIALYZER, and the sample is dialyzed against a water absorbing polymer. The dialysis membrane retains the proteins inside the dialysis bag or device and the sample is concentrated as the water is pulled across the dialysis membrane.

An ideal water absorbing polymer would be polyethylene glycol. G-Biosciences offers both a Concentrator Solution and a Concentrator Powder for this purpose. The other group has developed a Tube-O-Concentrator contains the liquid concentrator and dialysis devices for added convenience (https://www.gbiosciences.com/image/pdfs/protocol/786-610_protocol.pdf).

### C: Electrophoresis

Electrophoresis is a useful technique to separate mixtures of proteins based on their size, shape, charge and charge-to-mass ratio. Generally, it is primarily used as a pre-fractionating technique for one-dimensional separation. Some of the most common electrophoretic pre-fractionation methods include electrokinetic methods which rely on isoelectric focusing (IEF) steps. These methods allow the proteins to remain in their native conformation so they are ideally used when protein bioactivity must be preserved (Guttman et al. 2004; Jorgenson and Evans, 2004).

### D: Chromatography

Chromatography is commonly defined as a physical method for separating individual components (solutes) of a mixture between the stationary phase and the mobile phase. The differences in partition coefficients of the different molecules cause them to separate in the stationary phase.

Liquid chromatography (LC) separates different proteins according to their size, charge, hydrophobicity, or specificity, and is the most commonly used technique in proteome pre-fractionation. It can also be used to remove some interference substances (e.g. salts) which may have been carried through from previous enrichment steps.

Ion-exchange chromatography (IEX), which separates proteins according to their pI, is the most commonly used LC fractionation technique. Anion-exchange chromatography is ideally used in fractionating acidic proteins while cation-exchange chromatography is useful in fractionating basic proteins (Gomez-Ruiz et al. 2007a).

Reverse phase LC (RP-LC) separates proteins based on hydrophobicity. In RP-LC, the mobile phase is slightly more polar than the stationary phase. The hydrophobic molecules in the mobile phase are adsorbed in the stationary phase while the hydrophilic molecules are eluted with increasing concentration of an organic solvent (e.g. acetonitrile). RP-LC is widely used in combination with IEX and MS analysis as an alternative to 2D-PAGE technology.

Affinity chromatography (AC) utilizes highly specific biological interactions to investigate posttranslational modifications (e.g. glycosylation and phosphorylation). Since AC binds high abundance proteins to the column, it can be used to access low concentrated proteins in complex samples (Azarkan et al. 2007).

Immobilized metal affinity chromatography (IMAC) which is based on formation of coordinate bonds between basic groups on protein surface and metal ions, is mainly used for enriching phosphoproteins. Some of the disadvantages of using this technique includes the fact that there is minimal binding to Fe(III) or Ga(III) charged resins at neutral pH, and that using low-pH buffers may trigger protein denaturalization or precipitation in the column (Schmidt et al. 2007).

Size-exclusion chromatography (SEC) separates proteins according to their molecular mass. This technique can be performed under non-denaturing conditions so you can use it in studying protein complexes.

### SUMMARY OF THE INVENTION

Protein Concentration is an important step in protein extraction and purification. Large amounts of starting materials are generally used to acquire an adequate quantity of a protein of interest. Following protein extraction, the purified proteins need to be concentrated. The purpose of inventing of this device was to make an efficient tool with low cost to rapidly isolate and concentrate the secreted proteins/enzymes of microorganisms in high quality. By this device, the secreted proteins are concentrated by passing through a dialysis membrane at electrophoretic conditions.

### BRIEF DESCRIPTION OF DRAWINGS

FIG. 1 is a schematic diagram of protein electrodialysis (PED)

FIG. 2 displays the amount of total protein at top and bottom of the column, as well as inside the buffer of harvesting container at different time points.

FIG. 3 displays the amount of reduced sugars obtained from the decomposition of cellulose by the enzyme at top and bottom of the column, as well as inside the buffer of harvesting container at different time points.

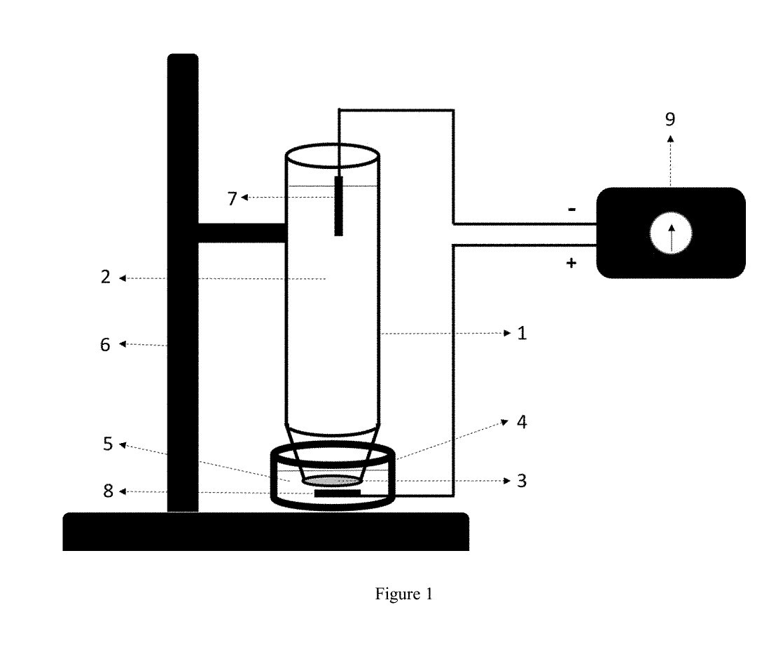

### LIST OF ALL ITEMS

The following is the list of all the components used in this invention as well as a brief description of each piece:
1 Column/main container: A 50 ml plastic column tube (Falcon, USA) selected as column
2 Centrifuged Liquid Culture Medium: The liquid culture medium inside the column has already been centrifuged at 2000 rpm for 5 min to remove any fungi mycelium/bacteria.
3 Dialysis Membrane: The bottom of the main container/column (1) was cut and covered with a sheet of plastic dialysis membrane (3) with molecular weight cut-off 12 KDa. The membrane was fixed using plastic glue.
4 Harvesting container: A plastic container was placed under the dialysis membrane (3).
5 The Buffer of Harvesting Container: The Harvesting Container (4) was filled with a buffer (e.g.Tris-Hcl pH=7).
6 Holder: A metallic backing holds the main container/column (1) and the harvesting container (4).
7 Anode: The negative electrode made of platinum was placed above the column
8 Cathode: The positive electrode made of platinum was placed in the harvesting container (4).
9 Power supply: The power supply was used to provide the electric energy needed for moving proteins across the column (1) and through the dialysis membrane (3).

### DETAILED DESCRIPTION OF SPECIFICATION

The subject of electrophoresis deals with the controlled motion of charged particles in electric fields. Since proteins are charged molecules, they migrate under the influence of electric fields. The electrophoresis mobility of a protein depends on its charge, size and shape.

For enrichment of the proteins; the liquid culture medium was centrifuged at 2000 rpm at 5 min to remove fungi mycelium/bacteria. Then as much as 50 ml of the centrifuged liquid culture medium (2) was poured into the main container (1), afterwards the electric charge (15 mA) was set in the device using the power supply (9). In this device, the electric field between the liquid culture medium (2) and the buffer inside the harvesting container (5) leads to the movement of protein molecules from the negative electrode (7) at top of the column to the positive electrode (8) in the buffer of the harvesting container (5). At the end of the procedure, as much as 3 ml of protein molecules would be concentrated in the buffer of the harvesting container (5).

### Reliability

To check the performance of the device and the results obtained from, we designed an experiment detailed as follows:

Fungal isolate: Isolation of Trichoderma harzianum was grown on PDA medium and stored at 4° C.

Culture medium: carboxy methyl cellulose (CMC) medium containing 0.05 g FeSO4 7H2O, 0.25 g MnSO4 H2O, 0.25 g CoC12, 0.25 g ZnSO4, 0.25 g (NH4)2SO4, 2 g KH2PO4, 0.25 g MgSO4 7 H2O, 0.4 g CaCl2, 0.3 g urea, 0.2 ml Tween 80 and 10 g Carboxy Methyl Cellulose per liter were prepared for cellulose degradation experiments. Fifty milliliter of broth was distributed in 250 ml Erlenmeyer and then media was autoclaved at 120° C. for 20 min.

Inoculation and sampling: Flask was inoculated with 1 ml spore suspension in three replicates for each species. The flasks were treated at 25° C. for 31 days and then medium culture centrifuged at 2000 rpm for 5 min.

Protein electrodialysis (PED): Fifty ml of the centrifuged culture medium was poured into the column (main container) and the anode electrode was fixed in the liquid culture medium (2) at top of the column (1); while the cathode electrode was put into the buffer of the harvesting container (5). Then the power supply was set at 15 miliA.

Quantification of Protein and the reduced sugar: To check the performance of the device, the amount of cellulose enzyme and the related substrate (reduced sugar assay) were measured at top of the column, bottom of the column and inside the harvesting container. To do that, sampling (50 μl) was done at each 15 min time point for 120 min. Released fungal extracellular proteins and produced sugars concentrations were determined using Bradford method and Arsenate-Molybdate reagent assay, respectively (Bradford, 1976; Kossem and Nannipieri, 1995).

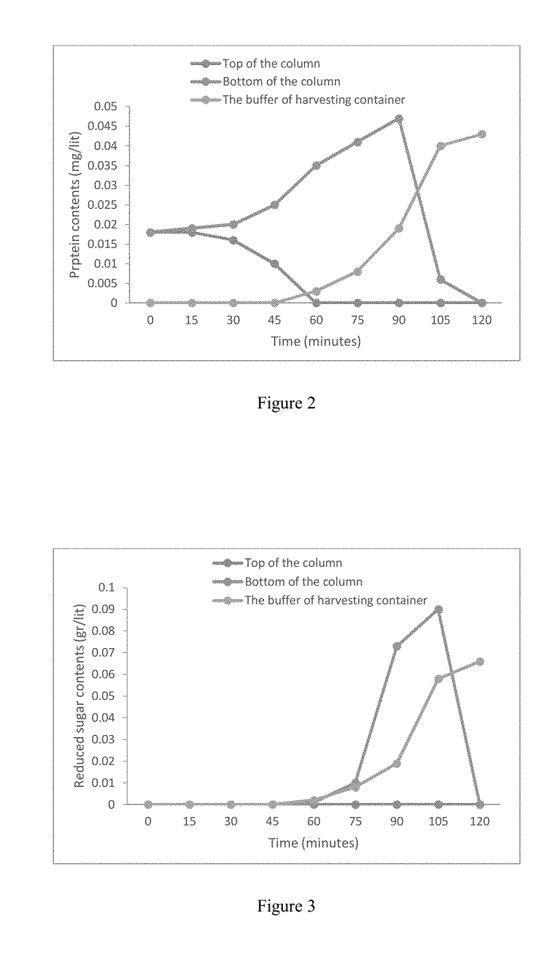

Results: Based on our results the protein content gradually decreased at top of the column; while increased at down of the column throughout 90 min after beginning the Protein electrodialysis (PED). On the other hand, the amount of the protein increased in the harvesting container at 45 min after the beginning of the experiment (FIG. 2). Accordingly, the amount of the reduced sugar was increased at the bottom of the column and inside the harvesting container throughout 45 min post beginning of Protein electrodialysis (PED) (FIG. 3).

The PED device of this invention is a low cost and user friendly laboratory instrument that concentrates secreted proteins/enzymes obtained from microorganisms such as filamentous fungi and bacteria. It can be used to concentrate proteins/enzymes secreted by microorganisms which have been grown in liquid culture medium. Also proteins inside any liquid culture media can be concentrated by PED after centrifugation at 2000 rpm for 5 min. Up to 50 ml of liquid culture medium can be used to concentrate the secreted proteins/enzymes using PED. It is suggested to use PED at cold room temperature, where it can be used in a variety of small and large biotechnology laboratories.

1- A Protein Electrodialysis (PED) device comprising:
A holder;
a main container;
a harvesting container;
a dialysis membrane and power supply;
wherein said holder is a metallic backing, holding said main container and said harvesting container in a desired position with respect to each other; wherein said main container comprises an open end and a closed end, wherein said closed end is cut open and covered with a sheet of plastic of said dialysis membrane using plastic glue; and wherein said harvesting container is a plastic container placed facing and under said dialysis membrane.

2- The device of claim 1, wherein said membrane has a molecular weight cut off of 12 KDa.

3- The device of claim 2, wherein said main container comprises a predetermined quantity of an enriched protein (centrifuged liquid culture medium); and said harvest container comprises a buffer.

4- The device of claim 3, wherein said power supply comprises an anode and cathode end; wherein one end (said anode) is located inside said main container touching said culture medium and the other end (said cathode) is located inside said harvest container being in contact with said buffer; wherein when said power supply is turned on, a second quantity of molecules of said enriched protein move from said main container into said harvesting container.

5- The device of claim 4, wherein said predetermined quantity of said enriched protein is at most 50 ml and said second quantity is at least 3 ml.

6- The device of claim 5, wherein said culture medium comprises but not limited to carboxy methyl cellulose (CMC) medium containing 0.05 g FeSO4 7H2O, 0.25 g MnSO4 H2O, 0.25 g CoC12, 0.25 g ZnSO4, 0.25 g (NH4)2SO4, 2 g KH2PO4, 0.25 g MgSO4 7 H2O, 0.4 g CaCl2, 0.3 g urea, 0.2 ml Tween 80 and 10 g Carboxy Methyl Cellulose per liter.

7- The device of claim 6, wherein said buffer comprises Tris-Hcl with pH=7.
